(19) United States
(12) Patent Application Publication
(10) Pub. No.: US 2021/0205113 A1
(43) Pub. Date: Jul. 8, 2021

(54) MATERNITY WAIST-BELLY SUPPORT BELT

(71) Applicant: CRYSTAL APPAREL LTD., HONG KONG (CN)

(72) Inventor: Chifai WONG, HONG KONG (CN)

(21) Appl. No.: 16/074,055

(22) PCT Filed: Jan. 22, 2018
(86) PCT No.: PCT/CN2018/073675

§ 371 Date: Jul. 30, 2018

(51) Int. Cl. A61F 5/03

(52) U.S. Cl. CPC A61F 5/03

(57) ABSTRACT

Disclosed is a shaped seamless maternity waist-belly support belt (100) which is formed by a circular knitting machine knitting yarns made from textile materials such as nylon and spandex and is seamless and barrel-shaped, which is characterized in that: the support belt comprises a plurality of portions, and these portions are located at different parts of the support belt and have respective specially-designed knit structures. The present disclosure further provides a garment including the shaped seamless waist-belly support belt (100) attached to a waistband of a bottom garment. The waist-belly support belt according to the present disclosure is seamless and barrel-shaped, without seams and without concavity or convexity in an outer surface or an inner surface, and provides high comfort. Meanwhile, since the waist-belly support belt has different knit structures at different parts to suit the growing belly shape, it can provide support for the pregnant woman's waist and back and reduce pelvic pains.

### FIELD OF THE DISCLOSURE

The present disclosure relates to a maternity waist-belly support belt (commonly called bellyband), and specifically relates to a seamless barrel-shaped knitted waist-belly support belt. The present disclosure further relates to a garment to which the maternity waist-belly support belt is attached.

### BACKGROUND OF THE DISCLOSURE

As well known, women are confronted with a series of physical discomforts during pregnancy. For example, the belly of a pregnant woman grows as the fetus develops. Pains increase in the back, waist and pelvis and therefore affect the pregnant woman's movement capability and cause inconvenience to daily life. Traditionally, maternity garments are dresses or knitted pants which are loose in size to provide comfort to the wearer but with no special support to the belly. There are maternity garments and maternity belts available for accommodating the growing size of the belly and supporting the wearer's back to reduce the pelvic pain. Some maternity garments are equipped with stretchable belts which allow pants, shorts or skirt to be left open at the waist and use the stretchable belt connecting directly or indirectly by buttons to the waistband of such bottom garments. The problem of this type of maternity garment is that they are in two pieces and not flexible in wearing. Such maternity garments equipped with a waist-belly support belt are usually tight to the body, have seams and their belly areas are not air permeable. There are also some special maternity garments each are provided with a waist-belly support belt which is adjustable according to the belly size. However, these waist-belly support belts are usually adjustable according to the belly shape as using an elastic material and they are not engineered to fit the shape of the belly. Therefore, this type of maternity garments cannot provide proper support for the belly and cannot reduce the pelvic pains.

There are maternity belts and abdominal support belts available and designed to reduce the back and pelvic pains. Most of such belly bands are a piece of stretchy material with Velcro closures or hook-and-eye closures. The Velcro closure usually becomes loose after wearing for some time. These maternity belts should not be worn for a whole day as this will make the muscles dependent on the extra support and won't fix the issues already presented. Besides, such maternity belt is usually worn under clothes and thus looks bulky and causes discomfort to the wearer, in particular during the summer time.

Jeans are a wardrobe staple for most women. It is usually manufactured with less elastic woven denim fabric, which is not comfortable enough for pregnant women and thus normally not considered for maternity apparel. Pregnant women will then not be able to continue to wear jeans during the pregnancy period.

### SUMMARY OF THE DISCLOSURE

To solve the above problems existing in the prior art, the present disclosure provides a waist-belly support belt, which has a specially-designed knit structure, may be attached to a waistband of a bottom garment, and provides different functions and support for a woman during pregnancy period.

According to an aspect of the present disclosure, there is provided a waist-belly support belt which is formed by knitting elastic yarns made from textile materials and is seamless and barrel-shaped, wherein the support belt is a shaped structure comprising at least four portions, namely, at least comprises an upper edge, a front panel, a rear panel and a lower edge, wherein the upper edge is located at a top of the waist-belly support belt; the lower edge is located at the lowest end of the waist-belly support belt; the front panel is located in the front of the waist-belly support belt with an upper side of the front panel being adjoined with the upper edge; the rear panel is located in the rear of the waist-belly support belt; and the knit structures of the front panel and the rear panel are different and different from the knit structure of the upper edge or the lower edge.

Preferably, in the waist-belly support belt, the upper edge and the lower edge each are a Rib (Aligned Repeat) Knit Structure; the front panel is formed by a Mesh Design Miss Stitch Combo Alternated Repeat Knit Structure or Small Holes Design Knit Structure; the rear panel is formed by a Miss Stitch Aligned Repeat Knit Structure.

Preferably, the waist-belly support belt further comprises a support portion and a triangular portion, and the support portion is located between the front panel and the lower edge and surrounds a lower side of a connection portion and a lower side of a narrow portion; the triangular portion is a triangular zone compliant with ergonomics, and located between the support portion and the rear panel and at the waist of both sides of the waist-belly support belt with its widest bottom located at the side waist and with its acute angle tapered towards a center back.

Preferably, the support portion is formed by a Miss Stitch Alternated Repeat Knit Structure, and the triangular portion is formed by a Miss Stitch Aligned Repeat Knit Structure.

Preferably, the waist-belly support belt further comprises an elongated connection portion and a narrow portion, wherein both sides and a lower side of the front panel are adjoined with the connection portion so that the connection portion surrounds the both sides and lower side of the front panel; the narrow portion includes two strip-shaped zones adjoined behind the connection portion, adjoined behind the connection portion between the front panel and the rear panel, and adjoined before the triangular portion.

Preferably, the connection portion and the narrow portion each are Miss Stitch Alternated Repeat Knit Structure.

Preferably, in another aspect, in the waist-belly support belt, the upper edge is a 5×1 Rib (Aligned Repeat) Knit Structure; the front panel is a Mesh Design Miss Stitch Combo Alternated Repeat Knit Structure; the connection portion is a 3×1 Miss Stitch Alternated Repeat Knit Structure; the narrow portion is a 3×1 (2 courses) Miss Stitch Alternated Repeat Knit Structure; the support portion is a 3×1 (3 courses) Miss Stitch Alternated Repeat Knit Structure; the triangular portion is a 2×2 (9 courses) Miss Stitch Aligned Repeat Knit Structure; the rear panel is a 1×1 (10 courses) Miss Stitch Aligned Repeat Knit Structure; the lower edge is a 3×1 Rib (Aligned Repeat) Knit Structure.

Preferably, for the materials used in the waist-belly support belt are 90-96% nylon and 4-10% spandex. More preferably, content materials being used in the waist-belly support belt are 92% nylon, and 8% spandex.

Preferably, the textile materials for the waist-belly support belt can further comprise polymer fibers such as viscose rayon or nylon, natural fibers or regenerated fibers.

Preferably, the textile materials for the waist-belly support belt can further comprise special featured or special functions polymer fibers.

In addition, the present invention, provides a garment comprising the waist-belly support belt according any of the above aspects the lower edge of the waist-belly support belt is attached to a waistband of a bottom garment so that the waist-belly support belt and the bottom garment become one piece.

Preferably, the above mentioned garment can be attached is not limited to stitching, spinning, heat fusion, button, snap fastener, velcro or zippers.

Preferably, the bottom garment to be attached can be pants, shorts or skirts.

Preferably, the pants in the garments mentioned are jeans.

The maternity waist-belly support belt according to the present disclosure is seamless and barrel-shaped, no side seams and without concavity and convexity in an outer surface and an inner surface, and provides great comfort. Also, the waist-belly support belt has different knit structure at different parts to suit the swelling belly shape, provide support for the pregnant woman's waist and back and reduce pelvic pains. In addition, the waist-belly support belt according to the present disclosure may be attached to various bottom garments to make the pregnant woman keep fashionable and feel comfortable, thereby substantially improving problems caused by conventional maternity belts.

### BRIEF DESCRIPTION OF THE DRAWINGS

FIGS. 1a and 1b are a front view and a rear view in which a seamless barrel-shaped knitted waist-belly support belt according to the present disclosure is attached.

FIGS. 2a, 2b and 2c are respectively a front view, a rear view and a side view of a waist-belly support belt according to a first embodiment of the present disclosure.

FIG. 3 is a longitudinal sectional view from center front to center back of the waist-belly support belt according to a first embodiment of the present disclosure.

FIG. 4 is a front view of each knit structure of a waist-belly support belt according to a second embodiment of the present disclosure.

FIGS. 5a, 5b, and 5c are respectively a perspective view, a front view and a rear view of the waist-belly support belt of the present disclosure attached to bottom garments.

### DETAILED DESCRIPTION OF PREFERRED EMBODIMENTS

The present disclosure will be described in detail according to specific embodiments, wherein the same reference numbers denote the same parts.

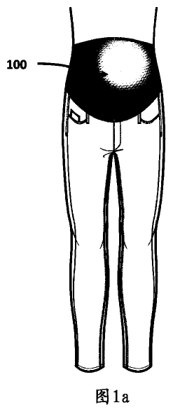

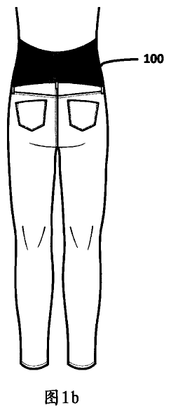

FIGS. 1a and 1b respectively show a front view and a rear view of a pregnant woman wearing a bottom garment to which a waist-belly support belt 100 according to the present disclosure is attached, wherein a shaped seamless barrel-shaped knitted waist-belly support belt 100 according to the present disclosure wraps the pregnant woman's waist and belly.

A first embodiment is described below.

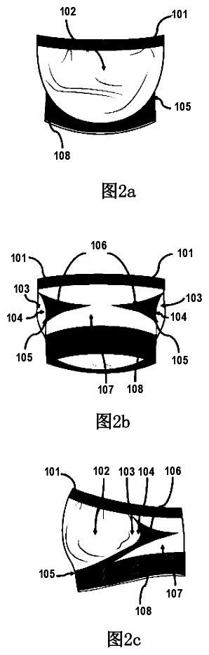

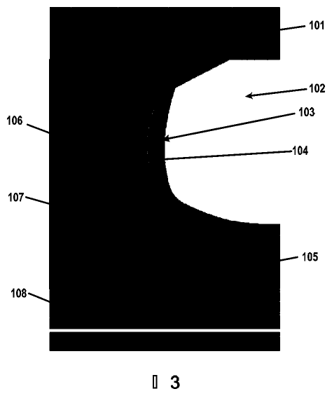

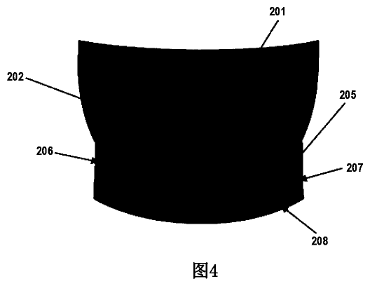

A structure, a manufacturing method and characteristics of the shaped seamless barrel-shaped knitted waist-belly support belt according to the first embodiment of the present disclosure will be described in detail in combination with FIGS. 2a-c and FIG. 3, wherein FIGS. 2a, 2b and 2c are respectively a front view, a rear view and a side view of the shaped seamless barrel-shaped knitted waist-belly support belt according to the first embodiment of the present disclosure. FIG. 3 is a longitudinal sectional view from center front to center back of the shaped seamless barrel-shaped knitted waist-belly support belt according to the first embodiment of the present disclosure, showing details of knit structures at different parts, wherein the left side of the view is back, and the right side of the view is front.

The waist-belly support belt 100 is formed by a circular knitting machine knitting elastic yarns made from textile materials of Nylon and Spandex, wherein nylon accounts for 90-96%, preferably 92% and Spandex accounts for 4-10%, preferably 8%. With the circular knitting design, a seamless waist-belly support belt may be formed so that the whole product does not have any seams and does not make the pregnant woman feel uncomfortable.

Different parts of the shaped seamless barrel-shaped knitted waist-belly support belt according to the present disclosure use different knit structures. Even though the fetus moves in the belly and makes the belly deform, the elastic structure can still adjust the shape of the waist-belly support belt to conform to the deformation of the belly so that the wearer does not feel uncomfortable, meanwhile the pregnant woman's back can be supported to reduce the pains in the waist.

The waist-belly support belt 100 comprises an upper edge 101, a front panel 102, a connection portion 103, a narrow portion 104, a support portion 105, a triangular portion 106, a rear panel 107 and a lower edge 108. These portions have respective different knit stitches. Since the support belt is knitted as a whole, boundaries where the portions are connected to one another are not clear, and no seams are formed. Knit structures of the portions and corresponding characteristics and functions are described in detail below.

The upper edge 101 of the waist-belly support belt is a 5×1 Rib (Aligned Repeat) Knit Structure with course 1: 5×1 (miss+knit stitch), course 2: all knit. The top of the waist-belly support belt forms a structure of an elastic band. The Miss Stitch structure is composed of stretch yarns of #2030 nylon covered yarn and 210D bare spandex yarn, ensuring good shape retention and perfect stretchiness for comfort, durability, fit and security. This elastic band structure also helps the shaped seamless barrel-shaped knitted waist-belly support belt fit comfortably over the belly and prevents the belt from sliding down.

The front panel 102 of the waist-belly support belt is located in the front of the support belt, covers the pregnant woman's belly, and uses a Mesh Design Miss Stitch Combo Alternated Repeat Knit Structure with course 1: 1×1 (miss+knit stitch), Course 2: 1×3 (miss+knit stitch) alternate with course 1, course 3: 1×1 (miss+knit stitch) alternate with course 1, course 4: all knit, forming the front potion of the waist-belly support belt. An upper side of the front panel is adjoined with the upper edge 101 and covers comfortably over the belly. This multiple stitches combination offer comprehensive protection to a pregnant woman. The mesh stitch structure provides breathability, and the alternate (miss+knit stitch) structure embraces the belly to provide better breathability during summer. This Miss Stitch Combo can also be replaced by other Small Holes Design Knit Structure.

The connection portion 103 of the waist-belly support belt is an elongated shape, and forms a rim surrounding the front panel 102 except the top. The connection potion 103 forms an enclosure only at both sides and the lower side of the front panel 102. The connection portion 103 uses a 3×1 Miss Stitch Alternated Repeat Knit Structure with course 1: 3×1 (miss+knit stitch), course 2: all knit. This is the first level of the intricate stitches structure. The stitch tension is the smallest but larger than the tension of the front panel 102 so that the pressure is delivered to the back.

The narrow portion 104 of the waist-belly support belt is adjoined behind the connection portion 103 at both sides of the support belt and then adjoined before the triangular portion 106. The narrow portion uses a 3×1 (2 courses) Miss Stitch Alternated Repeat Knit Structure with courses 1 & 2: 3×1 (miss+knit stitch), course 3: all knit. This structure adds one more (miss+knit stitch) course to the 3×1 Miss Stitch (Alternated Repeat) Knit Structure. It is the second level of the intricate stitch structure and has a higher power of pressure delivery to the waist and the back.

The support portion 105 of the waist-belly support belt uses a 3×1 (3 courses) Miss Stitch Alternated Repeat Knit Structure with courses 1 to 3: 3×1 (miss+knit stitch), course 4: all knit. This structure adds one more (miss+knit stitch) course to the 3×1 (2 courses) Miss Stitch Alternated Repeat Knit Structure, and it is the third level of the intricate stitch structure. Since the fetus's growth during pregnancy causes the size of the belly to change, the support portion 105 provides appropriate stretchiness from time to time in response to the change, and provides a certain upward supporting force.

The triangular portion 106 of the waist-belly support belt is a triangular zone compliant with ergonomics, and located at the waist of both sides of the waist-belly support belt with its widest bottom located at the side waist and its acute angle tapered towards the center back. The triangular portion 106 uses a 2×2 (9 courses) Miss Stitch Aligned Repeat Knit Structure with courses 1 to 9: 2×2 (miss+knit stitch), courses 10 to 11: all knit. The 2×2 (9 course) 2 needles (miss+knit stitch) in aligned repeat provide the highest tightness to the intricate knit structure. The triangular zone of the triangular portion 106 compliant with the ergonomics delivers the pressure transferred from the front side of the body to the center of the back of the body. The two triangular zones compliant with the ergonomics at the waist side grip the waist firmly with appropriate elasticity for waist movement.

The rear panel 107 of the waist-belly support belt is located at a rear side of the waist-belly support belt, and it is the widest part surrounding the belly which joints at the back of the waist. The rear panel 107 employs a 1×1 (10 courses) Miss Stitch Aligned Repeat Knit Structure with courses 1 to 9: 1×1 (miss+knit stitch), course 10: all knit. The stitch structure here is the densest and tightest amongst all, may provide a strong supporting force, and is pivotal for the waist and back support and protection.

The lower edge 108 of the waist-belly support belt is located at the lowermost end of the waist-belly support belt, and similar to the upper edge 101, employs a 3×1 Rib (Aligned Repeat) Knit Structure with course 1: 3×1 (miss+knit stitch), course 2: all knit. The lower edge is used to connect the waist-belly support belt to the waistband of the jeans, meanwhile enables the waistband of the jeans to fit close to the waist and prevents from slipping off.

As clearly shown from FIG. 3, the upper edge 101 is the top of the waist-belly support belt, is barrel-shaped, and functions to keep the shaped seamless barrel-shaped knitted waist-belly support belt at a proper position upon wearing and prevent slipping. The lower edge 208 is also barrel-shaped and may be connected to the top of the bottom garment. The front panel 102 forms the front portion of the waist-belly support belt and provides flexibility for the belly movement and breathability for the belly. A series of different knit structures are used between the connection portion 103 and the rear panel 207 to form one piece to deliver the pressure to the back and provide support for the waist and back to reduce the pregnant woman's pelvic pains.

A second embodiment is described below.

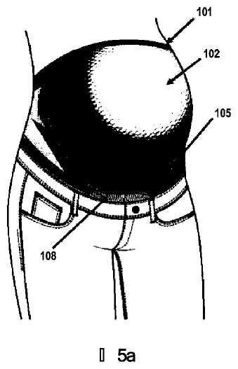

FIG. 4 is a front view of different knit structures of portions of a shaped seamless barrel-shaped knitted waist-belly support belt according to a second embodiment of the present disclosure. The implementation mode of the embodiment is substantially the same as the first embodiment, and the difference only lies in that as compared with the first embodiment, in the second embodiment the connection portion and the narrow portion are not provided, and the support belt in the second embodiment only comprises an upper edge 201, a front panel 202, a support portion 205, a triangular portion 206, a rear panel 207 and a lower edge 208, wherein the upper edge 201 is located at the top of the waist-belly support belt 100; the lower edge 208 is located at the lowermost end of the waist-belly support belt; the front panel 202 is located in the front of the waist-belly support belt, an upper side of the front panel 202 is adjoined with the upper edge 201, a lower side is adjoined with the support portion 205, and both sides are adjoined with the triangular portion 206; the support portion 205 is located between the lower side of the front panel 202 and the lower side of the triangular portion 206 and the upper side of the lower edge 208; the triangular portion 206 is a triangular zone compliant with ergonomics, and located at the waist of both sides of the waist-belly support belt with its widest bottom located at the side waist and with its acute angle tapered towards the center of the back; the rear panel 207 is located at a rear side of the waist-belly support belt. The above portions have the same knit structures, characteristics and functions as the corresponding portions in the first embodiment, which will not be detailed any more.

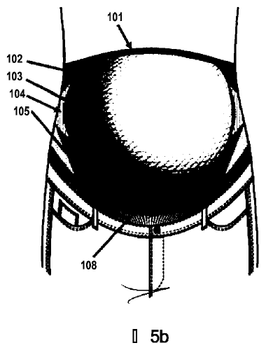

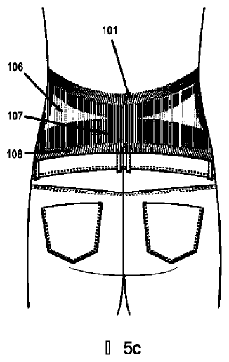

FIGS. 5a, 5b, and 5c are respectively a perspective view, a front view and a rear view of the shaped seamless barrel-shaped knitted waist-belly support belt of the present disclosure attached to bottom garments. Certainly, the shaped seamless barrel-shaped knitted waist-belly support belts in the first and second embodiments may both be attached to bottom garments. Usually, pregnant woman's bottom garments employ a low-waist design, and may accommodate increasingly larger belly during the pregnancy. Furthermore, the knit structure of the bottom of the waist-belly support belt of the present disclosure may provide smooth connection between the waist-belly support belt and the bottom garments. The connection manners of the waist-belly support belt and the bottom garments include but are not limited to sewing, spinning, heat fusion, button, snap fastener, Velcro or zipper. The bottom garments include but are not limited to pants, shorts and skirts. The pants may be jeans. The bottom garments may be woven or knitted garments.

The shaped seamless barrel-shaped knitted waist-belly support belt according to the present disclosure may be produced by using a Santoni knitting machine (further details available on www.santoni.com) which provides the seamless feature to minimize any discomforts caused by traditional maternity belts. Different knit stitches have been used in different parts of the shaped seamless barrel-shaped knitted waist-belly support belt to maintain comfort to the growing belly as well as to provide support to the waist and the back of the pregnant woman to reduce pelvic pain.

The top of the shaped seamless barrel-shaped knitted waist-belly support belt uses nylon covered yarns and spandex yarns, and knitted with a 5×1 Miss Stitch structure into an elastic band with elasticity, and has an excellent bouncing performance and tightness and ensures good comfort and avoids sliding off. The center front part of the shaped seamless barrel-shaped knitted waist-belly support belt covers the belly and is knitted from a Mesh Design Miss Stitch Combo. This part offers a comprehensive protection to the wearer's belly. The mesh design provides breathability and flexible movement for belly. The parts next to center front alongside of the waist towards the back and the lower part of the belly are knitted by a series of intricate stitches with different level of stitch tension from slack to tight. These intricate stitches are arranged in 4 levels of tightness which help to deliver the pressure to the back. A special stitch structure at a position next to the waist forms an engineered triangular pattern. The special stitch structure has an extremely tight tension and grips the waist firmly. The so doing also provides extra support for the bottom of the belly. The knitted stitches of the part at the bottom of the belly towards the center back area are the densest and tightest among all which is pivotal for the back support and protection.

Materials used in the present disclosure comprise nylon and spandex, providing properties such as comfort, durability, fitness and support to the pregnant woman. The spandex is Lycra®. Materials for knitting may further include polymeric fibers such as polyester fiber or soft nylon. Other yarns may also be of different contents with special functions and features such as hollow polyester fiber to keep warm and dry, or channelled-surfaced polyester fiber to keep cool and dry. Yarns made from different materials with various properties can also fulfill the purpose of back support (compression), comfort (flexibility and stretch; warmth or cooling). Examples of such yarns include yarns made from special featured polymer fibers such as Coolmax® (available from Invista™) which can be used for providing moisture wicking effect. Polymer fibers with special functions may also be included, for example, yarns like Thermolite® (available from Invista™) can provide warmth but light weight. Other natural fibers such as cotton or wool, regenerated fibers such as viscose rayon or Lyocell can be used in combination with the above polymer fibers to create natural feel fabric while maintaining the required properties of support and comfort. The elasticity ratio of the yarns themselves provides the product with elasticity of different degrees to suit the pregnant woman's belly size in different pregnancy phases.

The present disclosure is described in detail above according to the specific embodiments of the present disclosure. However, those skilled in the art should understand the present disclosure is not limited to said specific embodiments, and instead, variations and improvements may be performed without departing from the essence of the present disclosure and fall within the scope as defined in the appended claims.

### LISTS OF REFERENCE NUMBERS

100 waist-belly support belt
101, 102 upper edge
102, 202 front panel
103 connection portion
104 narrow portion
105, 205 support portion
106, 206 triangular portion
107, 207 rear panel
108, 208 lower edge

1. A waist-belly support belt which is formed by knitting elastic yarns made from textile materials and is seamless and barrel-shaped, wherein the support belt is a shaped structure comprising at least four portions, namely, at least comprising an upper edge, a front panel, a rear panel and a lower edge, wherein the upper edge is located at a top of the waist-belly support belt; the lower edge is located at the lowest end of the waist-belly support belt; the front panel is located in the front of the waist-belly support belt with an upper side of the front panel being adjoined with the upper edge; the rear panel is located in the rear of the waist-belly support belt; and the knit structures of the front panel and the rear panel are different and different from the knit structure of the upper edge or the lower edge.

2. The waist-belly support belt according to claim 1, further comprising a support portion and a triangular portion, the support portion being located between the front panel and the lower edge and surrounding a lower side of a connection portion and a lower side of a narrow portion; the triangular portion, is a triangular zone compliant with ergonomics, and located between the support portion and the rear panel and at the waist of both sides of the waist-belly support belt with its widest bottom located at the side waist and with its acute angle tapered towards a center back.

3. The waist-belly support belt according to claim 2, further comprising an elongated connection portion and a narrow portion, wherein both sides and a lower side of the front panel are adjoined with the connection portion so that the connection portion surrounds the both sides and lower side of the front panel; the narrow portion includes two strip-shaped zones adjoined behind the connection portion, adjoined behind the connection portion between the front panel and the rear panel, and adjoined before the triangular portion.

4. The waist-belly support belt according to claim 1, wherein the upper edge and lower edge each are formed by Rib (Aligned Repeat) Knit Structure; the front panel is formed by a Mesh Design Miss Stitch Combo Alternated Repeat Knit Structure or Small Holes Design Knit Structure; the rear panel is a formed by Miss Stitch Aligned Repeat Knit Structure.

5. The waist-belly support belt according to claim 2, wherein the upper edge and lower edge each are a Rib (Aligned Repeat) Knit Structure; the front panel is a Mesh Design Miss Stitch Combo Alternated Repeat Knit Structure or Small Holes Design Knit Structure; the support portion is a Miss Stitch Alternated Repeat Knit Structure; the triangular portion and the rear panel are Miss Stitch Aligned Repeat Knit Structures.

6. The waist-belly support belt according to claim 3, wherein the upper edge and lower edge each are a Rib (Aligned Repeat) Knit Structure; the front panel is a Mesh Design Miss Stitch Combo Alternated Repeat Knit Structure or Small Holes Design Knit Structure; the connection portion, the narrow portion and the support portion each are a Miss Stitch Alternated Repeat Knit Structure; the triangular portion and the rear panel are Miss Stitch Aligned Repeat Knit Structures.

7. The waist-belly support belt according to claim 5, wherein the upper edge is a 5×1 Rib (Aligned Repeat) Knit Structure; the front panel is a Mesh Design Miss Stitch Combo Alternated Repeat Knit Structure; the support portion is a 3×1 (3 courses) Miss Stitch Alternated Repeat Knit Structure; the triangular portion is a 2×2 (9 courses) Miss Stitch Aligned Repeat Knit Structure; the rear panel is a 1×1 (10 courses) Miss Stitch Aligned Repeat Knit Structure; the lower edge is a 3×1 Rib (Aligned Repeat) Knit Structure.

8. The waist-belly support belt according to claim 6, wherein the upper edge is a 5×1 Rib (Aligned Repeat) Knit Structure; the front panel is a Mesh Design Miss Stitch Combo Alternated Repeat Knit Structure; the connection portion is a 3×1 Miss Stitch Alternated Repeat Knit Structure; the narrow portion is a 3×1 (2 courses) Miss Stitch Alternated Repeat Knit Structure; the support portion is a 3×1 (3 courses) Miss Stitch Alternated Repeat Knit Structure; the triangular portion is a 2×2 (9 courses) Miss Stitch Aligned Repeat Knit Structure; the rear panel is a 1×1 (10 courses) Miss Stitch Aligned Repeat Knit Structure; the lower edge is a 3×1 Rib (Aligned Repeat) Knit Structure.

9. The waist-belly support belt according to claim 1, wherein in textile materials, nylon accounts for 90-96%, and spandex accounts for 4-10%.

10. The waist-belly support belt according to claim 1, wherein materials are 92% nylon, and 8% spandex.

11. The waist-belly support belt according to claim 1, wherein the textile materials further comprise polymer fibers such as viscose rayon or soft nylon, natural fibers or regenerated fibers.

12. The waist-belly support belt according to claim 1, wherein the textile materials further comprise special featured or special functions polymer fibers.

13. A garment, comprising the waist-belly support belt according to claim 1, wherein the lower edge of the waist-belly support belt is attached to a waistband of a bottom garment so that the waist-belly support belt and the bottom garment become one piece.

14. The garment according to claim 13, wherein the attachment manners include sewing, spinning, Velcro or zipper.

15. The garment according to claim 13, wherein the bottom garment is pants, shorts or skirts.

16. The garment according to claim 15, wherein the pants are jeans.
